(19) United States
(12) Patent Application Publication
(10) Pub. No.: US 2021/0233500 A1
(43) Pub. Date: Jul. 29, 2021

(54) System and Method for Dynamically Focusing an Information Handling System Display Screen Based on User Vision Requirements

(71) Applicant: Dell Products L.P., Round Rock, TX (US)

(72) Inventor: Alok Sinha, Bangalore (IN)

(73) Assignee: Dell Products L.P., Round Rock, TX (US)

(21) Appl. No.: 16/775,399

(22) Filed: Jan. 29, 2020

(51) Int. Cl. G09G 5/373; A61B 3/032

(52) U.S. Cl. CPC G09G 5/373; G09G 2340/045; G09G 2354/00; A61B 3/032

(57) ABSTRACT

A system of one or more computers is configured to perform particular operations or actions by virtue of having software, firmware, hardware, or a combination of them installed on the system that dynamically focuses the display of an information handling system screen based on user vision requirements. At least one embodiment includes receiving a vision power requirement for a user and determining a distance between a facial target area of the user and the display screen of the information handling system. An image magnification for the display screen is set based on the vision power requirement and the distance between the facial target area of the user and the display screen as measured by the distance sensor. The magnification value for images on the display screen is dynamically updated based on changes in the distance between the facial target area of the user in the display screen.

### BACKGROUND OF THE INVENTION

### Field of the Disclosure

The present disclosure relates to information handling systems. More specifically, embodiments of the disclosure relate to a system and method for dynamically focusing an information handling system display screen based on user vision requirements.

### Description of the Related Art

As the value and use of information continues to increase, individuals and businesses seek additional ways to process and store information. Options available to users include information handling systems. An information handling system generally processes, compiles, stores, and/or communicates information or data for business, personal, or other purposes thereby allowing users to take advantage of the value of the information. Because technology and information handling needs and requirements vary between different users or applications, information handling systems may also vary regarding what information is handled, how the information is handled, how much information is processed, stored, or communicated, and how quickly and efficiently the information may be processed, stored, or communicated. The variations in information handling systems allow for information handling systems to be general or configured for a specific user or specific use such as customer record management, business projection analysis, etc. In addition, information handling systems may include a variety of hardware and software components that are configured to process, store, and communicate information and may include one or more computer systems, data storage systems, and networking systems.

### SUMMARY

A system of one or more computers can be configured to perform particular operations or actions by virtue of having software, firmware, hardware, or a combination of them installed on the system that in operation causes or cause the system to dynamically focus the display of an information handling system screen based on user vision requirements. At least one embodiment is directed to a computer-implemented method for operating a display screen of an information handling system, the method may include: receiving a vision power requirement for a user; determining a distance between a facial target area of the user and the display screen of the information handling system using a distance sensor disposed proximate the display screen; setting an image magnification for the display screen based on the vision power requirement and the distance between the facial target area of the user and the display screen of the information handling system as measured by the distance sensor; automatically tracking the distance between the facial target area of the user and the display screen using the distance sensor; and automatically updating the image magnification for the display screen in response to changes in the distance between the facial target area and the display screen as measured by the distance sensor. Other embodiments of this aspect include corresponding computer systems, apparatus, and computer programs recorded on one or more computer storage devices, each configured to perform the actions of the methods.

At least one embodiment is directed to a system having a processor; a data bus coupled to the processor; and a non-transitory, computer-readable storage medium embodying computer program code, the non-transitory, computer-readable storage medium being coupled to the data bus, the computer program code interacting with a plurality of computer operations and may include instructions executable by the processor and configured for: receiving a vision power requirement for a user; determining a distance between a facial target area of the user and the display screen of the information handling system using a distance sensor disposed proximate the display screen; setting an image magnification for the display screen based on the vision power requirement and the distance between the facial target area of the user and the display screen of the information handling system as measured by the distance sensor; automatically tracking the distance between the facial target area of the user and the display screen using the distance sensor; and automatically updating the image magnification for the display screen in response to changes in the distance between the facial target area and the display screen as measured by the distance sensor.

At least one embodiment is directed to a non-transitory, computer-readable storage medium embodying computer program code, the computer program code may include computer executable instructions configured for: receiving a vision power requirement for a user; determining a distance between a facial target area of the user and the display screen of the information handling system using a distance sensor disposed proximate the display screen; setting an image magnification for the display screen based on the vision power requirement and the distance between the facial target area of the user and the display screen of the information handling system as measured by the distance sensor; automatically tracking the distance between the facial target area of the user and the display screen using the distance sensor; and automatically updating the image magnification for the display screen in response to changes in the distance between the facial target area and the display screen as measured by the distance sensor.

### BRIEF DESCRIPTION OF THE DRAWINGS

The present disclosure may be better understood, and its numerous objects, features and advantages made apparent to those skilled in the art by referencing the accompanying drawings. The use of the same reference number throughout the several figures designates a like or similar element.

FIG. 1 is a generalized illustration of an information handling system that is configured to implement certain embodiments of the system and method of the present disclosure.

FIG. 2 depicts a position that may be assumed by a user during initialization operations.

FIG. 3 depicts a user at two different horizontal distances with respect to a central portion of a display screen.

FIG. 4 depicts differences between the distance of a target facial region, such as the user's eyes, and the screen when the user tilts their head.

FIG. 5 depicts differences between the distance of a user's eyes and the screen when the user rotates their head.

FIG. 6 depicts a screen displaying the same image at two different magnification levels.

FIG. 7 is a flowchart showing exemplary operations that may be executed in certain embodiments of the disclosed system.

FIG. 8 is a flowchart showing exemplary operations that may be executed in certain embodiments of the disclosed system.

FIG. 9 shows exemplary locations for placement of the distance sensors.

FIG. 10 is a flowchart showing exemplary operations that may be executed by the disclosed system to allow the user to determine the vision power needed to correct the user's vision.

### DETAILED DESCRIPTION

Systems and methods are disclosed for dynamically focusing an information handling system display screen based on user vision requirements. In certain embodiments, a user provides a vision power requirement either directly or through an automated power selection initialization operation. The distance between a distance sensor disposed proximate the display screen and a facial target area of the user is employed to dynamically control display magnification values. As such, certain embodiments maintain a generally consistent vision power compensation for the user's vision requirements as the distance between the display screen and facial target area changes. In certain embodiments, the eyes of the user are used as the facial target area. In certain embodiments, an angle of the display screen with respect to the line of sight between the user and the display screen is also used in calculating the distance between the display screen and the facial target area.

For purposes of this disclosure, an information handling system may include any instrumentality or aggregate of instrumentalities operable to compute, classify, process, transmit, receive, retrieve, originate, switch, store, display, manifest, detect, record, reproduce, handle, or utilize any form of information, intelligence, or data for business, scientific, control, or other purposes. For example, an information handling system may be a personal computer, a network storage device, or any other suitable device and may vary in size, shape, performance, functionality, and price. The information handling system may include random access memory (RAM), one or more processing resources such as a central processing unit (CPU) or hardware or software control logic, ROM, and/or other types of non-volatile memory. Additional components of the information handling system may include one or more disk drives, one or more network ports for communicating with external devices as well as various input and output (I/O) devices, such as a keyboard, a mouse, and a video display. The information handling system may also include one or more buses operable to transmit communications between the various hardware components.

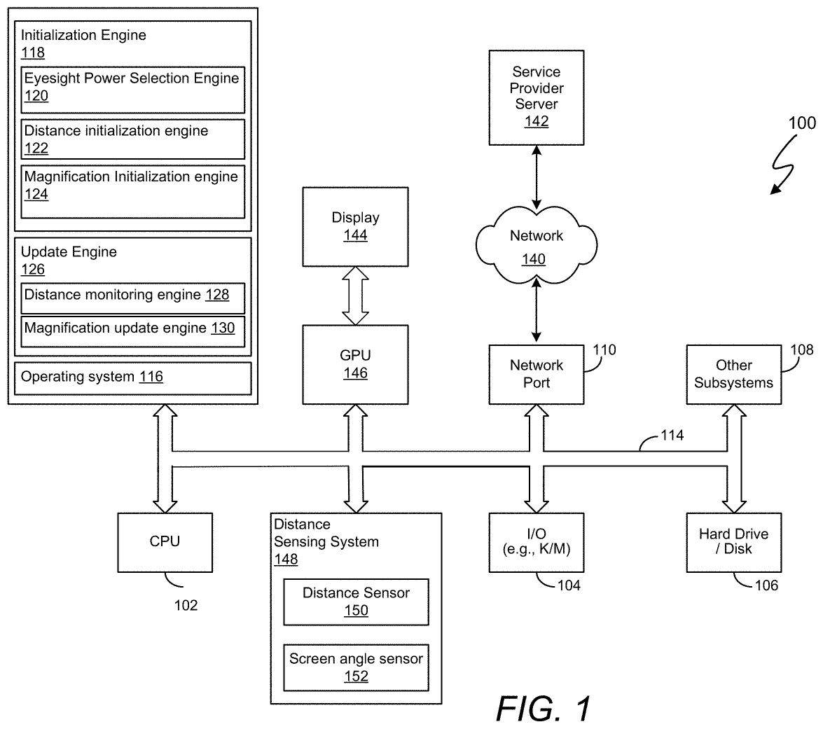

FIG. 1 is a generalized illustration of an information handling system 100 that is configured to implement certain embodiments of the system and method of the present disclosure. The information handling system 100 includes a processor (e.g., central processor unit or “CPU”) 102, input/output (I/O) devices 104, such as a display, a keyboard, a mouse, and associated controllers, a hard drive or disk storage 106, and various other subsystems 108. In various embodiments, the information handling system 100 also includes network port 110 operable to connect to a network 140, which is accessible by a service provider server 142. In certain embodiments, a user interacts with the various components and engines of the information handling system 100 through a user interface 138.

The exemplary information handling system 100 also includes a display 144, such as an LCD display, an LED display, or any display type suitable for displaying images to a user. Images may include any type of information that is display to a user to allow user to interact with the information handling system number 100. Such images may include pictures, wordprocessing documents, spreadsheets, multimedia, etc.

The display 144 is under the control of a graphics processing unit 146. In certain embodiments, commands may be issued to the graphics processing unit 146 over, for example, bus 114. Such commands may include commands that change the size and/or resolution of an image on the screen of display 144. As described herein, the size and/or resolution of images on the screen of display 144 may be adjusted to meet vision power requirements for a user.

The information handling system 100 likewise includes system memory 112, which is interconnected to the foregoing via one or more buses 114. System memory 112 may be local memory, remote memory, memory distributed between multiple information handling systems, etc. System memory 112 further comprises an operating system 116 and, in various embodiments may also comprise other software modules and engines configured to implement certain embodiments of the disclosed system.

In the example shown in FIG. 1, memory 112 includes an initialization engine 118. In certain embodiments, the initialization engine 118 executes operations that are used to configure the initial parameters used to place the magnification of the display screen image in an initial state in which the images on the screen are focused for the user. Certain embodiments of the initialization engine 118 include an eyesight power selection engine 120, a distance initialization engine 122, and a display screen magnification initialization engine 124. The eyesight power selection engine 120 is configured to set a vision power factor for the user. The vision power factor, in turn, is used to calculate a corresponding magnification value for increasing and/or decreasing the magnification of images on the screen of display 144. In certain embodiments, the vision power factor is entered directly by a user. In certain embodiments, the vision power factor is selected during an initialization routine in which the user is presented with a test image at varying magnifications until the user selects a magnification that the user feels adjusts the image to meet the user's vision power factor.

The exemplary initialization engine 118 also includes a distance initialization engine 122. In certain embodiments, the distance initialization engine 122 receives information from a distance sensing system 148 and calculates the distance between a facial feature of the user and a distance sensor 150 disposed proximate the display screen. In certain embodiments, the distance sensor 150 resides in the same plane as the plane of the display 144, and the plane of the screen of display 144 is generally parallel to the plane of the facial target area. However, in certain embodiments, the plane of the screen of display 144 is disposed at an angle with respect to the plane of the facial target area. Examples of displays that may be at an angle with respect to the facial target area include displays supported by an adjustable monitor assembly, displays used on laptop computer systems, etc. Accordingly, the distance sensing system 148 in certain embodiments may include a screen angle sensor 152. The screen angle sensor 152 is configured to detect the angle between the plane of the display screen and another plane, such as the plane of the surface upon which the displaying screen rests (e.g., desktop surface, the lap of a user, etc.) Certain embodiments may use the value of the screen angle to provide a more granular measure of the distance between the target facial area of the user and the screen of the display 144.

Certain embodiments of the initialization engine 118 may include a magnification initialization engine 124. In certain embodiments, the magnification initialization engine 124 is configured to determine the initial magnification value that will be used to compensate for deficiencies in the user's vision. At least one embodiment, the initial magnification M is determined as:

1/di=D−1/do and M=−di/do

where:
D=vision power;
di=distance between the display screen and the virtual image projected on the screen;
do=distance between the display screen and the target facial area; and
M=screen image magnification.

Certain embodiments of the disclosed system include an update engine 126 configured to dynamically adjust the magnification of images on the screen of the display 144 in response to changes in the distance between the target facial area of the user and the screen of the display 144. In the example shown in FIG. number one, the update engine 126 includes a distance monitoring engine 128, which monitors distance information provided by the distance sensing system 148. The distance information is provided to a magnification update engine 130, which determines a new magnification M′ determined as:

1/di′=D−1/do′ and M=−di′/do′

where:
D=vision power;
di′=the new distance between the display screen and the virtual image projected on the screen;
do′=the new distance between the display screen and the target facial area;
   and
M′=the new screen image magnification.

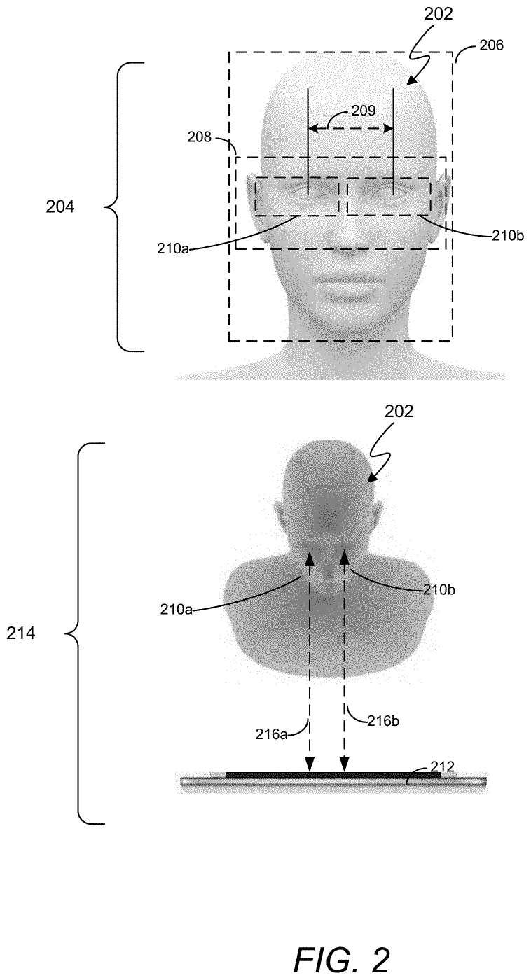

FIG. 2 depicts the position that may be assumed by a user 202 during initialization operations. In the example shown in FIG. 2, the head of the user 202 is vertically upright with both eyes forward. The A frontal view of the facial features of the user 202 is shown at 204. The frontal view 204 depicts various regions of the face that may be used as target facial features for purposes of measuring the distance between the user and the screen of the display. The depicted facial feature regions, in order of increasing granularity, include: all features in the frontal region 206 of the face, features in the mid-region 208 of the face, and individual eye regions 210a and 210b. In certain embodiments, selection of the target region 206, 208, and 210 that is to be used for distance measurements may vary depending on the distance and/or angle between the user and the screen of the display (e.g., regions having higher granularity are used at greater distances and regions having lower granularity are used at smaller distances).

An exemplary relationship between the position of the user 202 and the screen 212 of display 144 during initialization is shown at 214. In this example, the user 202 is positioned with both eyes facing the screen 212 so that the distances 216a and 216b are substantially equal. In this manner, embodiments of the disclosed system may generate an initial set of distance parameters from which an initial value for the magnification may be determined. In certain embodiments, facial recognition algorithms may be used during the initialization operations to guide the user to the desired initial relationship with the display screen. Also, certain embodiments may detect the distance 209 between a user's eyes for use in determining distances that are used to calculate the magnification when the user's head is rotated.

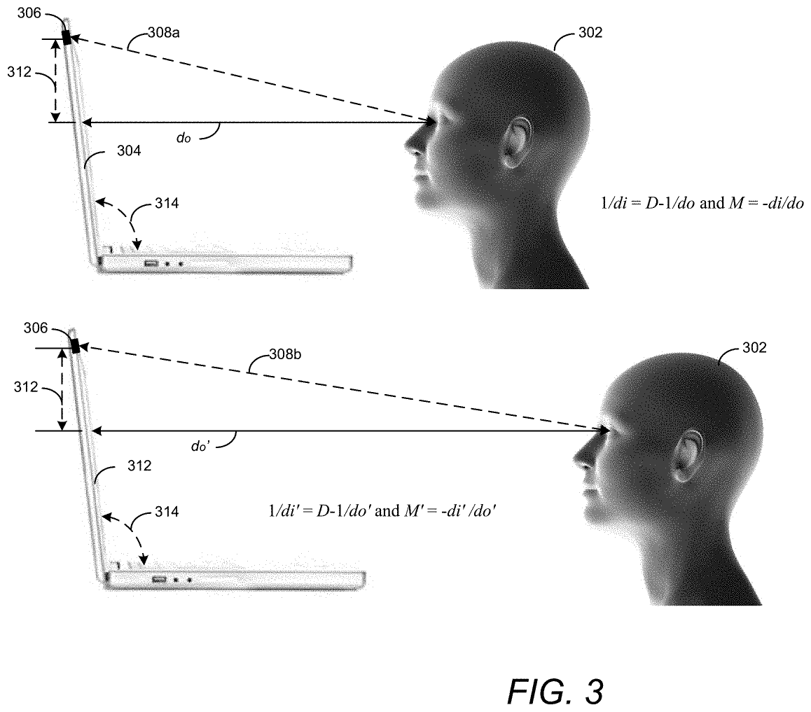

FIG. 3 depicts a user 302 at two different horizontal distances with respect to a central portion of display screen 304, shown here as the display of a laptop computer system. With the distance between the user and display screen (do) having a vision power D, the magnification M is determined as:

1/di=D−1/do and M=−di/do

FIG. 3 also depicts the user 302 at a distance from the display screen where do<do′. With the distance between the user and the display screen being do′ and the vision power D being constant for the user, the updated magnification M′ is determined as:

1/di′=D−1/do′ and M′=−di′/do′

In at least one embodiment, the distance sensor 306, such as a depth perceiving camera, is placed in a bezel of the display. As such, the distance sensor 306 does not directly measure the distance between the target facial area of the user 302 and the display screen 304. Rather, the distance sensor measures the distance 308a and 308b between the target facial area and sensor 306. In certain embodiments, a direct reading of the distances 308a and 308b may be used as the values for do and do′, respectively. However, certain embodiments may more accurately Determining distances do and do′ using parameters such as the distance 312 between sensor 306 and a central portion of the display screen 304, the value of angle 314, etc. In the example shown in FIG. 3, angle 314 corresponds to the angle between the plane of the keyboard and the plane of the display screen 312.

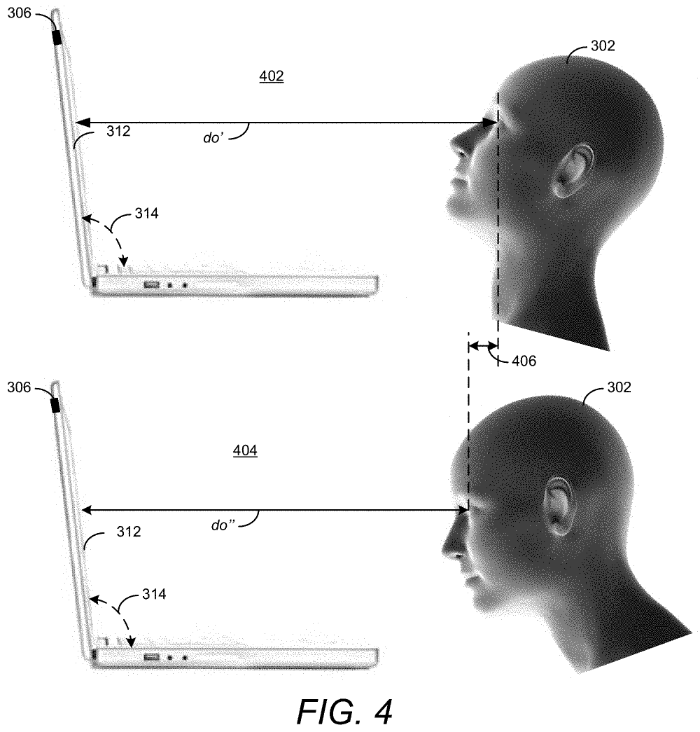

FIG. 4 depicts differences between the distance of a target facial region, such as the user's eyes, and the screen 312 when the user 302 tilts their head. In this example, the distance between the target facial region and the screen 312 has a value of do′ when the user tilts their head backward, and has a value of do″ when the user tilts their head forward. In this example, there is a change 406 in the distance between the target facial region and the screen as the user tilts their head between the angles shown in FIG. 4. Certain embodiments may increase the granularity of changes in magnitude by monitoring distances associated with head tilt.

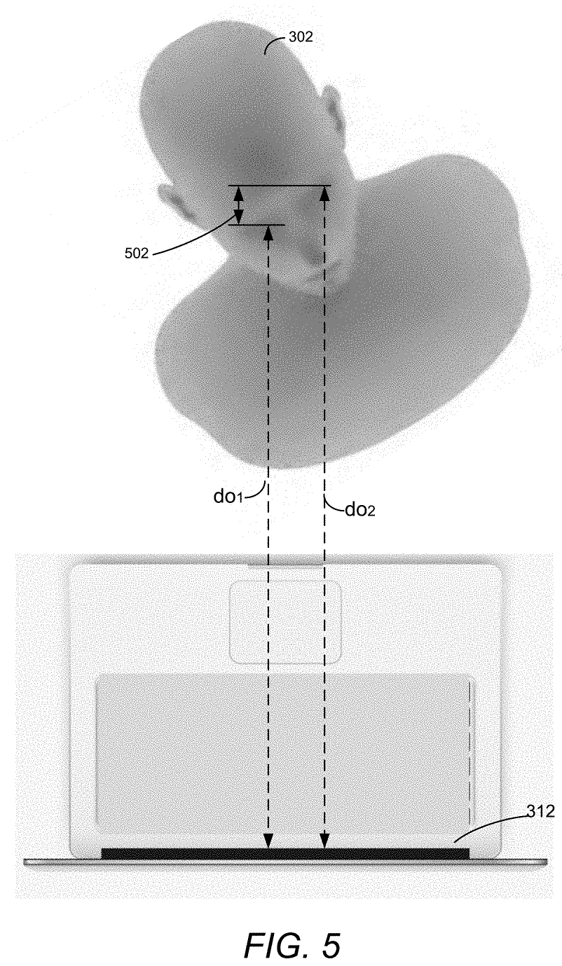

FIG. 5 depicts differences between the distance of a target facial region, such as the user's eyes, and the screen 312 when the user 302 rotates their head. In this example, the distance between the user's right eye and screen 312 is do₁, while the distance between the user's left eye and screen 312 is do₂ resulting in a distance difference shown at 502. In certain embodiments, the magnification is determined using do₁ so that the magnification is dependent on the nearest eye of the user. In certain embodiments, the magnification is determined using do₂ so that the magnification is dependent on the furthest eye of the user. In certain embodiments, the values of do1 and do2 are averaged so that the magnification is a compromise between magnifications associated with the nearest and furthest eye. Certain embodiments may increase the granularity of changes in magnitude by monitoring distances associated with head rotation.

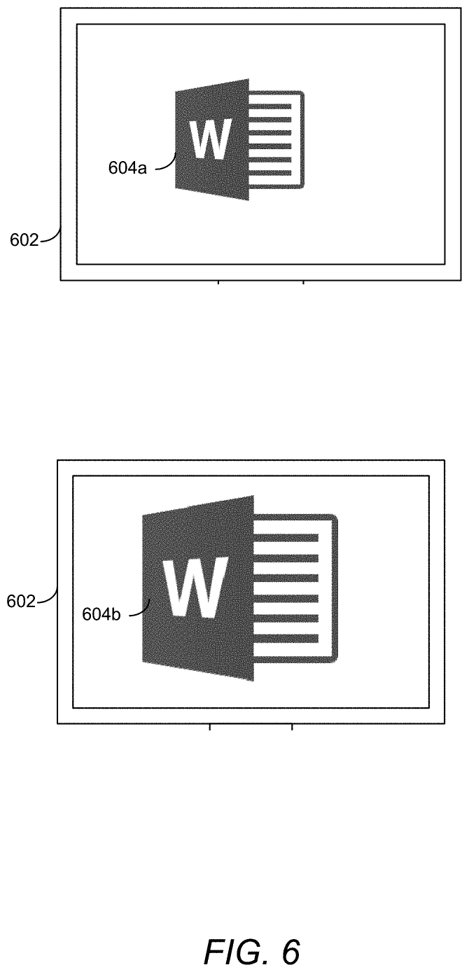

FIG. 6 depicts a screen displaying the same image at two different magnification levels. In this example, image 604b is presented on screen 602 at a larger magnification then image 604b. As noted above, the images may be wordprocessing documents, spreadsheets, pictorial images, video, etc.

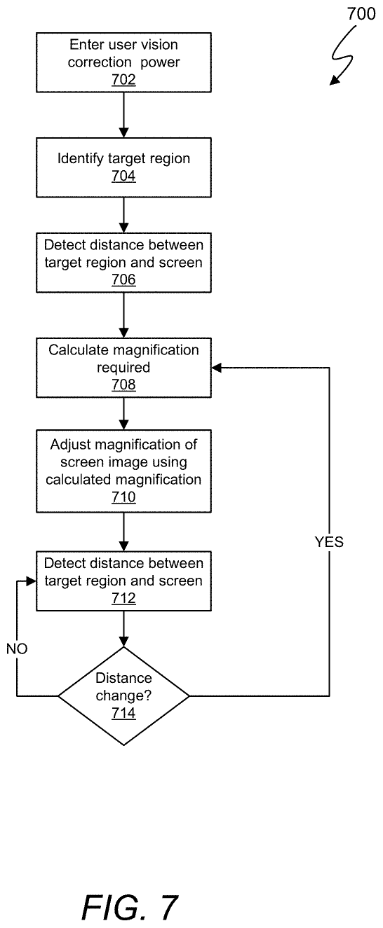

FIG. 7 is a flowchart 700 showing exemplary operations that may be executed in certain embodiments of the disclosed system. In this example, the user enters their vision correction power (D) at operation 702. The correction power D may be obtained from glasses prescribed by an optician, from the magnification power of non-prescription glasses, and/or determined through initialization operations described herein.

At operation 704, the target facial region that is to be used to determine the distance with the display screen is identified, and the distance between the target region and screen is detected at operation 706. Examples of such target facial regions are shown at the front facial view 204 shown in FIG. 2. Using the correction power D and distance between the target region and the screen detected at operation 706, the required magnification is calculated at operation 708. At operation 710, the screen image is adjusted using the calculated magnification. In certain embodiments, the adjustment magnification commands are issued to a graphics processing unit that controls the screen image to adjust the magnification for the calculated magnification value.

The disclosed system dynamically changes the magnification of the display screen using the distance between the target facial region and display screen. To this end, the distance between the target region and the screen is detected at operation 712, and a determination is made at operation 714 as to whether the distance has changed. If the distance has changed, a new magnification is calculated at operation 708 and the screen image is adjusted using the new magnification value. In certain embodiments, a new magnification value is only calculated if the distance change at operation 714 exceeds a predetermined threshold value. In certain embodiments, a hysteresis operation is executed to prevent abrupt changes in screen magnification.

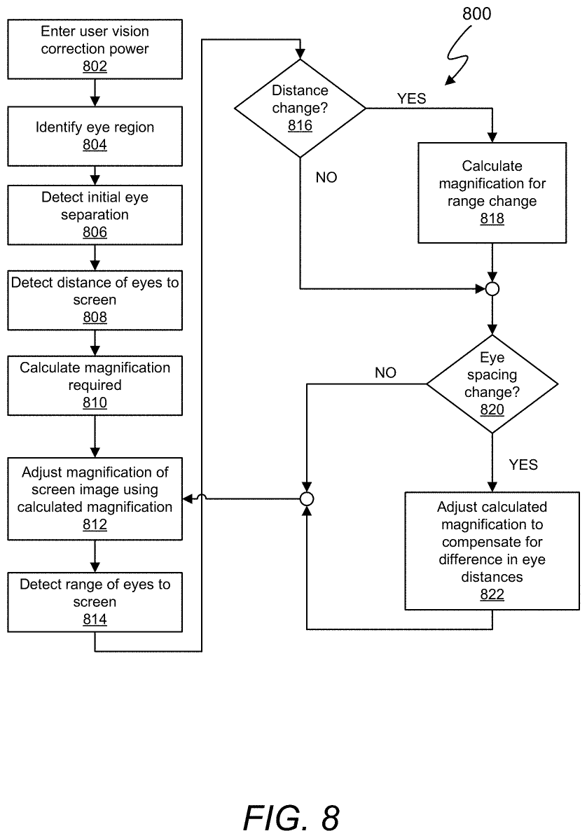

FIG. 8 is a flowchart 800 showing exemplary operations that may be executed in certain embodiments of the disclosed system. In the example shown in FIG. 8, the user's eyes are used as the target regions, and the operations shown in flowchart 800 include operations that may be executed when the user rotates their head.

At operation 802, the user enters the vision correction power D. At operation 804, the eye region is identified, and the distance between the eyes and screen is detected at operation 806. At operation 808, the initial eye separation is detected (see, e.g., FIG. 2). Using the correction power D and distance between the eye region and the screen detected at operation 804, the required magnification is calculated at operation 810. At operation 812, the screen image is adjusted using the calculated magnification. embodiments

The disclosed system dynamically changes the magnification of the display screen using the distance between the eye region and the display screen. To this end, the distance between the eye region and the screen is detected at operation 814 and a determination is made at operation 816 as to whether the distance has changed. If the distance has changed, a new magnification is calculated at operation 818. In certain embodiments, a new magnification value is only calculated if the distance change at operation 818 exceeds a predetermined threshold value. In certain embodiments, a hysteresis operation is executed to prevent abrupt changes in screen magnification.

At operation 820, a determination is made as to whether the eye spacing has changed due to rotation of the user's head. If the eye spacing has changed, the calculation at operation 818 is adjusted to compensate for differences in eye distances and the screen at operation 822, and the screen is adjusted to apply the new magnification using the calculated mate configuration at operation neural 812.

If the distance has not changed at operation 816, a check is made at operation 820 as to whether the eye spacing has changed. If the eye spacing has changed, the calculated magnification is currently used is adjusted to calculate a magnification that compensates for the difference in eye distances at operation 822. If neither the distance nor eye spacing have changed, the currently used magnification continues to be used until such time as either the distance changes at operation 816 and/or the eye spacing have changed at operation 820.

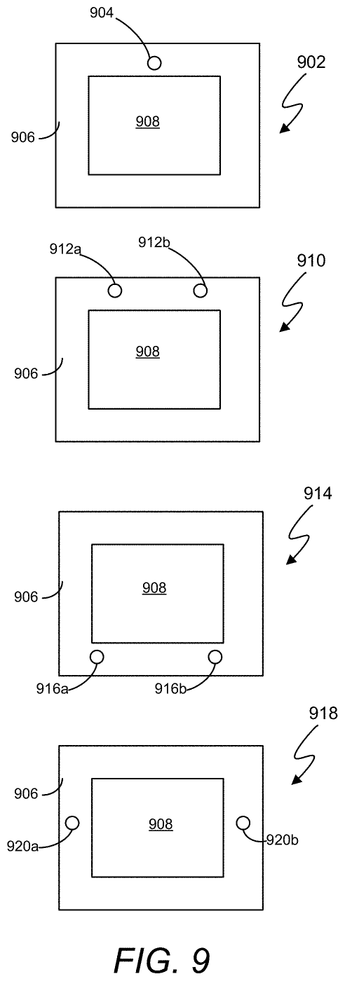

FIG. 9 shows exemplary locations for placement of the distance sensors. In configuration 902, the sensor 904 is disposed in, for example, a bezel 906 above the screen 908. In this configuration, the sensor 904 may be a camera capable of determining depth perception.

In configuration 910, at least two sensors 912a and 912b are disposed in the upper portion of the bezel 906 above the screen 908. In this configuration, sensors 912a and 912b may be two-dimensional cameras that are spaced from one another to allow binocular detection of depth.

In configuration 914, at least two sensors 916a and 916b are disposed in the bezel 906 at the lower portion the screen 908. In this configuration, sensors 916a and 916b may be two-dimensional cameras that are spaced from one another to allow binocular detection of depth.

In configuration 918, at least two sensors 920a and 920b are disposed in the bezel 906 at opposite sides of the screen 908. In this configuration, sensors 920a and 920b may be two-dimensional cameras that are spaced from one another to allow binocular detection of depth.

It will be recognized, in view of the teachings of the present disclosure, that various types of distance sensors and their corresponding placement may be used, the foregoing being non-limiting examples.

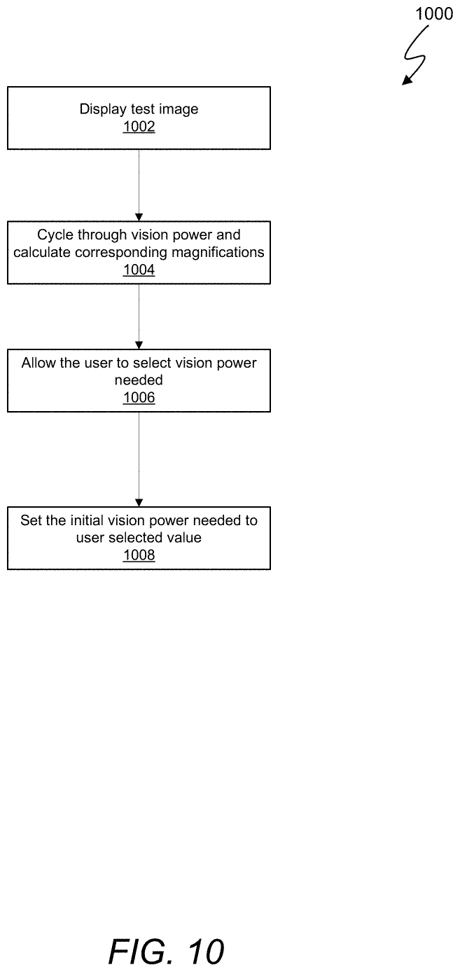

FIG. 10 is a flowchart 1000 showing exemplary operations that may be executed by the disclosed system to allow the user to determine the vision power needed to correct the user's vision. At operation 1002, a test image is displayed on the screen. The vision power used to magnify the display screen as it cycled through different power values at operation 1004. Cycling may continue until the user is satisfied with the magnification corresponding to the vision power at operation number 1006. The initial vision power value is set using the user-selected vision power value at operation 1008.

Embodiments of the disclosure are described with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer-readable memory that can direct a computer or other programmable data processing apparatus to function in a particular manner, such that the instructions stored in the computer-readable memory produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer-implemented process such that the instructions which execute on the computer or other programmable apparatus provide steps for implementing the functions/acts specified in the flowchart and/or block diagram block or blocks.

The disclosed system is well adapted to attain the advantages mentioned as well as others inherent therein. While the present invention has been depicted, described, and is defined by reference to particular embodiments of the invention, such references do not imply a limitation on the invention, and no such limitation is to be inferred. The invention is capable of considerable modification, alteration, and equivalents in form and function, as will occur to those ordinarily skilled in the pertinent arts. The depicted and described embodiments are examples only, and are not exhaustive of the scope of the invention.

1. A computer-implemented method for operating a display screen of an information handling system, the method comprising:
receiving a vision power requirement for a user;
determining a distance between a facial target area of the user and the display screen of the information handling system using a distance sensor disposed proximate the display screen and facing the facial target area;
setting an initial screen image magnification factor for the display screen based on the vision power requirement and the distance between the facial target area of the user and the display screen of the information handling system as measured by the distance sensor, where the initial screen image magnification factor is determined as
1/di=D−1/do and M=−di/do
where:
D=vision power requirement provided by the user,
di=distance between the display screen and a virtual image projected on the display screen,
do=distance between the display screen and the target facial area, and
M=screen image magnification factor;
automatically tracking the distance between the facial target area of the user and the display screen using the distance sensor; and
automatically updating the screen image magnification factor for the display screen in response to changes in the distance between the facial target area and the display screen as measured by the distance sensor, where updates to the screen image magnification factor are based on an updated screen image magnification factor determined as
1/di′=D−1/do′ and M′=−di′/do′
where:
di′=distance between the display screen and the virtual image projected on the display screen,
do′=new distance between the display screen and the target facial area, and
M′=updated screen image magnification factor based on do′.

2. The computer-implemented method of claim 1, wherein the facial target area includes the eyes of the user.

3. (canceled)

4. (canceled)

5. The computer-implemented method of claim 1, wherein the distance sensor is mounted in a bezel of the display screen.

6. The computer-implemented method of claim 1, wherein
the distance sensor includes at least one sensor selected from a group of sensors comprising:
a 3D camera;
a time-of-flight ranging sensor; and/or
multiple imaging devices spaced from one another and located proximate the display screen.

7. The computer-implemented method of claim 1, further comprising:
displaying a test image on the display screen;
allowing a user to cycle through multiple vision power values for the test image;
selecting, by the user, a visual power value based on the cycled images; and
setting initial image vision power parameters using the user-selected vision power value.

8. A system comprising:
a processor;
a display screen;
a data bus coupled to the processor; and
a non-transitory, computer-readable storage medium embodying computer program code, the non-transitory, computer-readable storage medium being coupled to the data bus, the computer program code interacting with a plurality of computer operations and comprising instructions executable by the processor and configured for:
receiving a vision power requirement for a user;
determining a distance between a facial target area of the user and the display screen of the information handling system using a distance sensor disposed proximate the display screen and facing the facial target area;
setting an initial screen image magnification factor for the display screen based on the vision power requirement and the distance between the facial target area of the user and the display screen of the information handling system as measured by the distance sensor, where the initial screen image magnification factor is determined as
1/di=D−1/do and M=−di/do
where:
D=vision power requirement provided by the user,
di=distance between the display screen and a virtual image projected on the display screen,
do=distance between the display screen and the target facial area, and
M=screen image magnification factor;
automatically tracking the distance between the facial target area of the user and the display screen using the distance sensor; and
automatically updating the screen image magnification factor for the display screen in response to changes in the distance between the facial target area and the display screen as measured by the distance sensor, where updates to the screen image magnification factor are based on an updated image magnification determined as
1/di′=D−1/do′ and M′=−di′/do′
where:
di′=new distance between the display screen and the virtual image projected on the display screen,
do′=new distance between the display screen and the target facial area, and
M′=updated screen image magnification factor based on do′.

9. The system of claim 8, wherein
the facial target area includes the eyes of the user.

10. (canceled)

11. (canceled)

12. The system of claim 8, wherein
the distance sensor is mounted in a bezel of the display screen.

13. The system of claim 8, wherein
the distance sensor includes at least one sensor selected from a group of sensors comprising:
a 3D camera;
a time-of-flight ranging sensor; and/or
multiple imaging devices spaced from one another and located proximate the display screen.

14. The system of claim 8, further comprising:
displaying a test image on the display screen;
allowing a user to cycle through multiple vision power values for the test image;
selecting, by the user, a visual power value based on the cycled images; and
setting initial image vision power parameters using the user-selected vision power value.

15. A non-transitory, computer-readable storage medium embodying computer program code, the computer program code comprising computer-executable instructions configured for:
receiving a vision power requirement for a user;
determining a distance between a facial target area of the user and the display screen of the information handling system using a distance sensor disposed proximate the display screen and facing the facial target area;
setting an initial screen image magnification factor for the display screen based on the vision power requirement and the distance between the facial target area of the user and the display screen of the information handling system as measured by the distance sensor, where the initial screen image magnification factor is determined as
1/di=D−1/do and M=−di/do
where:
D=vision power requirement provided by the user,
di=distance between the display screen and a virtual image projected on the display screen,
do=distance between the display screen and the target facial area, and
M=screen image magnification factor;
automatically tracking the distance between the facial target area of the user and the display screen using the distance sensor; and
automatically updating the screen image magnification factor for the display screen in response to changes in the distance between the facial target area and the display screen as measured by the distance sensor, where updates to the screen image magnification factor are based on an updated screen image magnification factor determined as
1/di′=D−1/do′ and M′=−di′/do′
where:
di′=new distance between the display screen and the virtual image projected on the display screen,
do′=new distance between the display screen and the target facial area, and
M′=updated screen image magnification factor based on do′.

16. The non-transitory, computer-readable storage medium of claim 15, wherein
the facial target area includes the eyes of the user.

17. (canceled)

18. (canceled)

19. The non-transitory, computer-readable storage medium of claim 15, wherein
the distance sensor is mounted in a bezel of the display screen.

20. The non-transitory, computer-readable storage medium of claim 15, wherein
the distance sensor includes at least one sensor selected from a group of sensors comprising:
a 3D camera;
a time-of-flight ranging sensor; and/or
multiple imaging devices spaced from one another and located proximate the display screen.
